(19) United States
(12) Patent Application Publication
(10) Pub. No.: US 2024/0307168 A1
(43) Pub. Date: Sep. 19, 2024

(54) PROPHYLACTICALLY IMPLANTABLE LUMEN-OBTURATED GRAFT

(71) Applicant: Paul Shridath Ramphal, Nassau (BS)

(72) Inventor: Paul Shridath Ramphal, Nassau (BS)

(21) Appl. No.: 18/608,117

(22) Filed: Mar. 18, 2024

Related U.S. Application Data
(60) Provisional application No. 63/490,555, filed on Mar. 16, 2023.

(51) Int. Cl. A61F 2/06; A61L 27/14

(52) U.S. Cl. CPC A61F 2/064; A61L 27/14

(57) ABSTRACT

A prophylactically implantable medical device for use for hemodialysis is disclosed. The device includes a tube graft and a removable plug, or obturator, that is co-extensive with the graft. The tube graft can be formed of a thermoplastic polymer while the obturator may be composed of a biologically inert, flexible, non-absorbable, and non-absorbent, material. The obturator is used to prevent proteinaceous body fluid from filling the entire lumen of the tube graft which has otherwise shown to eventually result in irreversible occlusion of the lumen as the proteinaceous fluid becomes organized into dense fibrous tissue.

### CROSS-REFERENCE TO RELATED APPLICATION

This application claims the benefit of priority of U.S. provisional application No. 63/490,555, filed Mar. 16, 2023, the contents of which are herein incorporated by reference.

### BACKGROUND OF THE INVENTION

The present invention relates to dialysis access and, more particularly, to a prophylactically implantable lumen-obturated graft for use for hemodialysis access.

The demand for dialysis services for people experiencing renal failure has been steadily growing worldwide. Long-term dialysis therapy may be a difficult experience for patients and for caregivers, often requiring multiple dialysis access procedures over time, in order to continue to be able to offer patients this life-saving therapeutic modality. In many cases, patients will at some point be users of the entire available range of options to facilitate hemodialysis, including temporary (uncuffed) indwelling dual lumen venous catheters (TUCVC), longer-term cuffed dual lumen venous catheters (CVC), arterio-venous fistulae (AVF) and arterio-venous grafts (AVG).

The most preferred option among healthcare professionals is the use of AVF, followed in preference by AVG, then CVC and lastly, TUCVC. This order of preference is based largely on the infection risk profile of each option, with AVF having the lowest such risk profile. This risk of infection increases significantly with each option down the preference scale. In addition, the use of either TUCVC or CVC carries unique risks of complications including, but not limited to, vascular injury at implantation leading to blood loss which may be sufficient to endanger the life of the patient, catheter lumen obstruction leading to equipment malfunction and inability to undergo effective hemodialysis, and recipient vessel fibrosis and chronic obstruction. In the latter situation, multiple interventional vascular procedures may be necessary to re-establish and maintain the venous lumen which can eventually end up with a loss of the venous pathway and a patient with internal jugular vein, subclavian vein, innominate vein, femoral vein, and/or superior or inferior vena caval obstruction and the attendant severe debilitating pathophysiological consequences of each of these or any combination of these sequelae.

Despite these known limitations, circumstances often dictate that a patient with newly diagnosed renal failure, whether acute or chronic, will have to begin their dialysis experience by using first a TUCVC, then a CVC, followed closely by surgical construction of an AVF, or even primary implantation of an AVG if no suitable veins for AVF are present anatomically. If an AVF is created, it is usually not available for use as a hemodialysis conduit for a period of at least six weeks, and more usually up to three months, as a period of “maturation” (cross-sectional luminal enlargement and wall thickening in response to blood flowing through the autologous venous conduit at arterial pressures and velocity) is necessary to allow the fistula to be located and safely cannulated (needled) by the dialysis technician. For AVG, this period is considerably shorter than needed for AVF. However even using so-called “immediate stick” grafts, a period of at least one week before attempted use is recommended, and many will recommend at least two weeks post-implantation before attempting to needle any graft for hemodialysis, to allow the development of a fibrous tissue enveloping “sheath” which is adherent to the outer wall of the AVG, which forms as a result of an expected and desired foreign body reaction by the patient's tissues to the material of the AVG (usually expanded polytetrafluoroethylene (PTFE), and occasionally DACRON™ cloth). At the completion of this biological process to envelop an AVG in dense fibrous tissue, the AVG is referred to as being “incorporated”. During the immediate and short-term period after either AVF or AVG creation or implantation, the patient will continue to require hemodialysis which will therefore need to be performed using one or more of the indwelling TUCVC or CVC devices, thus exposing the patient to the attendant short-term, and long-term risks of using these devices, until either maturation of the AVF or incorporation of the AVG, has occurred to the satisfaction of the surgeon and the dialysis staff.

In other scenarios, which are commonly seen, a patient with an AVF will experience a malfunction of the access, either due to thrombosis of the vessel or stenosis of a segment of the conduit or of the arterio-venous anastomosis, or because of a vascular steal phenomenon, or due to the development of a pseudoaneurysm or infection, or both, of a portion of the AVF. Any combination of such complications can and do occur, all resulting in an inability to utilize the AVF for a period. In the best case, an interventional endovascular procedure (dilation+/−stenting) of the stenotic portion of the AVF may be possible, which can often result in an AVF whose use may be immediately following the intervention. More often, the AVF would not be available for use immediately to allow for the healing of any surgical scars, new anastomoses, or the resolution of infection in surrounding tissues. For AVG, infection mandates removal of all prosthetic material involved, and pseudoaneurysm resection and repair often requires significant surgical incisions, tissue dissection, new anastomoses, and the need to have any new segments of prosthetic graft undergo the incorporation process prior to safe use for dialysis. In some situations, endovascular dilation+/−stenting of the graft to recipient vein anastomosis may be possible and may allow immediate or near immediate re-commencement of AVG use. Endovascular interventions are widely available in many developed nations, and often not available at all in the rest of the world and consequently to most the of world's dialysis patients.

Endovascular procedures involve expensive specialized techniques, expertise, and equipment, and in jurisdictions where such procedures are available, may be performed repeatedly and possibly frequently, as needed, on an individual patient, provided the procedures can be paid for through insurance or personal funds. Most often in the case of AVF or AVG malfunction, especially in places where interventional vascular procedures are not widely available, the patient will be required to undergo placement of a TUCVC or CVC to be able to continue hemodialysis treatment after any surgical intervention involving either de novo AVF or AVG, or repair or revision of an extant AVF or AVG.

As can be seen, there is a need for a prophylactically implantable lumen-obturated graft for use for hemodialysis access as described herein.

### SUMMARY OF THE INVENTION

With the discussion as outlined above in mind, it is apparent that it would be to the patient's advantage and to the caregiver's advantage (when not considering financial benefits to surgeons/interventionalists/for-profit institutions for interventional vascular procedures, but rather considering available operating theatre time, and personal time demands on patients and health care professionals created by urgent or emergent procedures arising from dialysis access malfunctions) to avoid, if at all possible, the need for TUCVC/CVC use, and to be able to return to using percutaneous needle cannulation techniques for dialysis, as soon as possible. In the case of AVF, it is also known that, eventually, most patients on long-term dialysis will utilize all available surgical sites for AVF, and most will then have to depend on AVG for percutaneous dialysis access.

In accordance with the present invention, an ideal solution to these problems, therefore, needs to enable truly immediate use of a percutaneous needling technique to continue hemodialysis without the need for even a short period of TUCVC or CVC use, for any of the possible scenarios outlined in the discussion above. The following description of the present invention addresses all these previously discussed issues.

In certain embodiments, a prophylactically implantable lumen-obturated graft (PILOG) is provided that is generally formed as a 6-millimeter (mm) or 8 mm diameter PTFE tube graft with a removable silicone (or similar material) obturator.

The following are certain advantageous and novel features of the present invention. Other features of the present invention will be readily apparent to those with skill in the art from the following disclosure. The PILOG is fully implantable in the way(s) any vascular prosthesis may be so implanted. The obturator may be left in situ for the entire time that the PILOG is in situ. The obturator is constructed with a method of grasping and removing it from the lumen of the PILOG, by means of an embedded knotted suture or some similar technique. The obturator is removed immediately prior to the establishment of the PILOG as an active dialysis conduit. Multiple PILOG grafts may be implanted in anticipation of future possible use. PILOG graft(s) may be implanted as a stand-alone procedure or as part of a procedure to establish an AVF of AVG for dialysis access. The obturator may be re-introduced into the PILOG or any other length of PTFE graft of appropriate luminal diameter to allow for maintenance of the lumen of the graft for possible future use while an alternative dialysis access route is being used. The rate of infection or other complication (e.g., pseudoaneurysm formation) associated with the PILOG can be expected to be equivalent to any other graft made of similar materials and being used for similar purposes. As a consequence of the prophylactic nature of its role as a dialysis vascular access conduit, the PILOG may be implanted in heretofore rarely or never utilized locations (e.g., the dorsal aspect of the arm or forearm) thus opening up a much larger variety of options for establishing chronic percutaneous dialysis access in these patients.

These and other features, aspects and advantages of the present invention will become better understood with reference to the following drawings, description, and claims.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, without departing from the scope of this disclosure.

FIG. 1 is a detail view of an end of an embodiment of the present invention;

FIG. 2 is a perspective view of the embodiment of the present invention;

FIG. 3 is a perspective view of the embodiment of the present invention, showing an obturator partially removed from a PTFE graft wall;

FIG. 4 is a perspective view of the embodiment of the present invention, showing the obturator fully removed from the PTFE graft wall;

FIG. 5 is a perspective view of the embodiment of the present invention, shown in use; and

FIG. 6 is a perspective view of three embodiments of the present invention, shown in use.

### DETAILED DESCRIPTION OF THE INVENTION

The subject disclosure is described with reference to the drawings, wherein like reference numerals are used to refer to like elements throughout. In the following description, for purposes of explanation, numerous specific details are set forth in order to provide a thorough understanding of the present disclosure such that one skilled in the art will be enabled to make and use the present invention. It may be evident, however, that the present disclosure may be practiced without some of these specific details. For the purpose of clarity, technical material that is known in the technical fields related to the present invention has not been described in detail so that the present invention is not unnecessarily obscured.

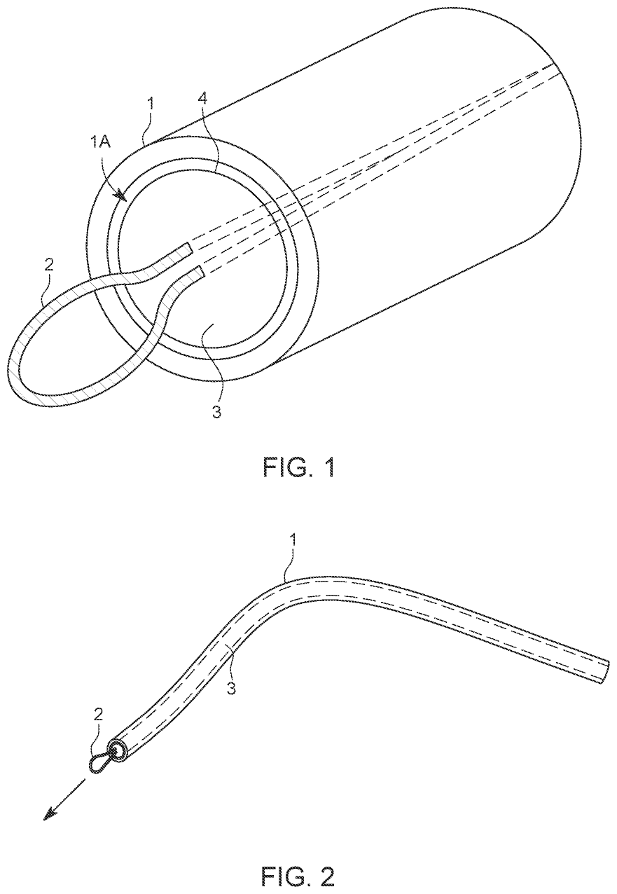

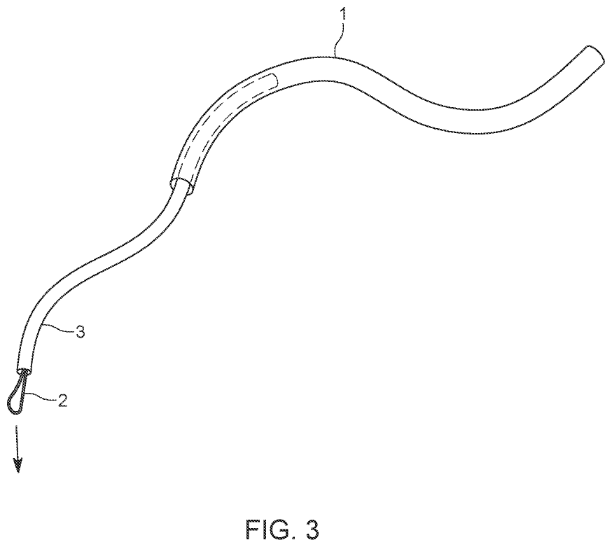

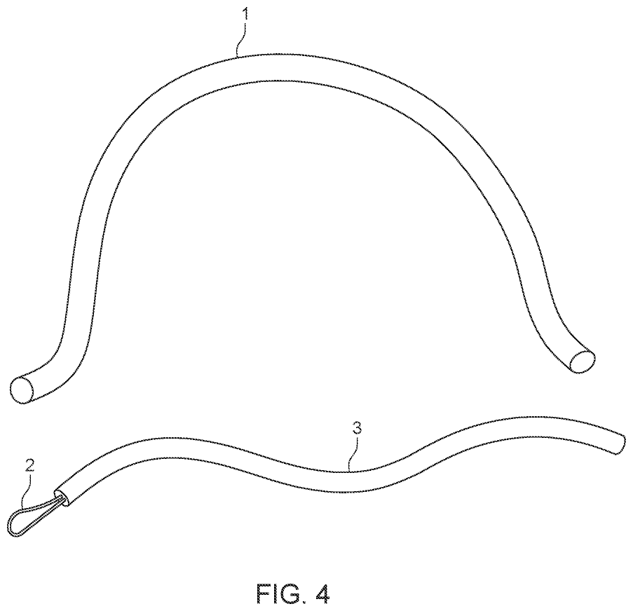

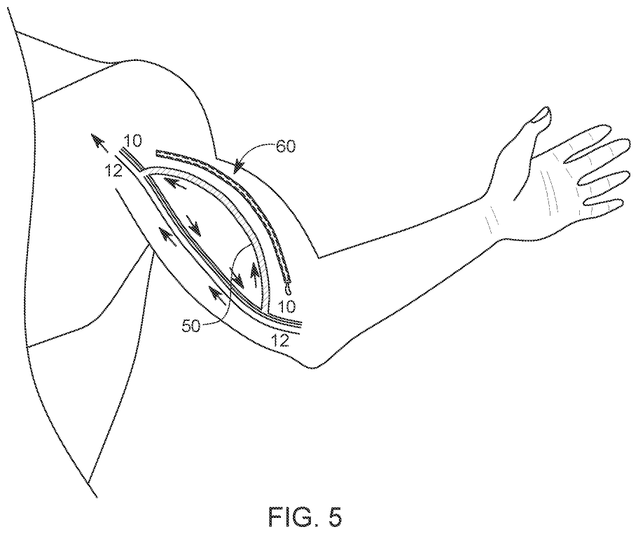

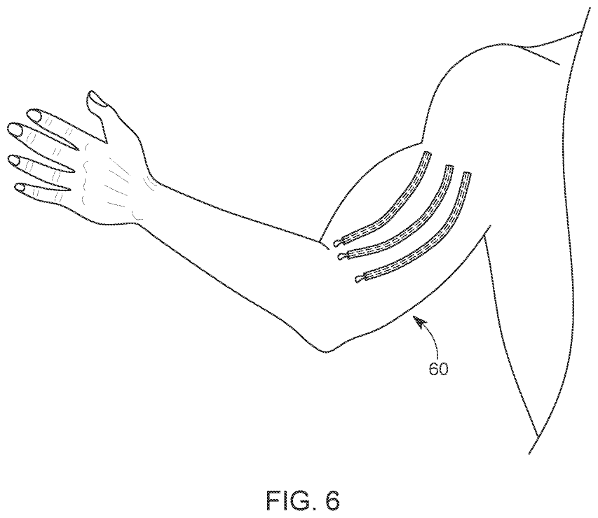

The present invention includes a medical device 60 (see FIG. 5), hereafter referred to as PILOG, and is generally depicted in FIGS. 1-4 and shown in use in FIGS. 5-6. The device 60 includes PTFE tube graft 1 and a removable plug 3, or obturator, that is co-extensive with the graft 1. The tube graft 1 may be 6 mm or 8 mm in lumen diameter, of variable total length, and similar in all respects to currently available products. The obturator 3 functions to completely occupy the entire length of the lumen 1A of the tube graft 1, with correspondent capillary space 4, while simultaneously maintaining the cylindrical cross-sectional profile of the graft 1 along its entire length.

In accordance with the present invention, the obturator 3 may be composed of a biologically inert, flexible, and non-absorbable, as well as being non-absorbent, material. Various materials may be used while staying in the spirit and scope of the present disclosure. For example, existing materials, such as an elastomer, e.g., medical implantation-grade silicone “rubber”, already widely in use in the field of implantable medical prostheses and devices, and materials that have yet to be created may be used.

The obturator 3 is primarily intended to prevent proteinaceous body fluid from filling the entire lumen 1A of the graft 1, which has otherwise shown to eventually result in irreversible occlusion of the lumen 1A as the proteinaceous fluid becomes organized into dense fibrous tissue. This phenomenon is observed regularly in AVG which are left in situ after thrombosis or after partial removal for malfunction. The phenomenon occurs even if the open ends of the tubular graft are ligated, as fluid will transude through the fibrillar matrix of the graft wall into the lumen 1A over time. The obturator 3 is left in place when the PILOG 60 is implanted and is removed by grasping (for example) a braided heavy nylon loop 2, or other suitable fibrous material, which is securely coupled to (e.g., by embedding within) the obturator 3 and placing traction on the loop 2. This traction causes the obturator 3 to slide out of the PTFE graft 1, leaving the lumen 1A fully patent along the length of the graft 1.

In one scenario, the PILOG 60 is designed to be implanted into a dialysis patient or a patient with renal insufficiency expected to eventually require dialysis for use as a future AVG, as shown in FIG. 5. For example, and as shown in FIG. 5, the PILOG 60 may be used as an AVG when either an AVF or conventional AVG 50 either must be abandoned or undergoes a revision procedure that temporarily renders the access unusable.

FIG. 5 demonstrate how the PILOG 60 may be implanted in much the same manner as a conventional AVG 50 would be. Specifically, the PILOG 60 may be tunneled in the subcutaneous tissues of the upper or lower limb utilizing a dedicated track and in a position that would allow for future anastomotic connection to the arterial 10 and venous 12 systems of the patient.

As those with skill in the art will appreciate, the PILOG 60 may be implanted as a part of the same procedure to create or implant the primary dialysis access AVF or AVG 50, or may be implanted by various methods. By way of example, it may be inserted via a stand-alone procedure, before or after the primary access procedure, and under local anesthesia only if preferred. However, after implantation, the PILOG 60 is simply left in situ, and undergoes the same foreign-body reaction as a standard AVG 50, leading to complete fibrous tissue incorporation after period of several weeks (see FIG. 5).

The lumen-occluding plug 3 is left in situ for the entire time that the PILOG 60 is implanted, which could conceivably be for years, until the graft 1 is required for use as a dialysis access conduit. At that time, a procedure may be performed, which starts with the removal of the plug 3 (via the loop 2) and continues with surgical techniques to connect the proximal and distal ends of the graft 1 with the arterial 10 and venous 12 systems of the patient. This may be accomplished by direct PILOG end to vessel-side or venous vessel-end with the now tubular PILOG 60, or by anastomosing either the proximal and/or distal stumps of the pre-existing PTFE graft (if not being deactivated because of, for example, sepsis) to the ends of the graft 1. It is also conceivable that after excision of a destroyed segment of an AVF that functional proximal and distal AVF stumps could be anastomosed to the graft 1, thereby creating a composite arterio-venous access conduit.

In another scenario, a repaired AVF or AVG (either by autologous venous interposition grafting, primary anastomosis, venous patching with autologous vascular tissue or synthetic material, synthetic conduit interposition in a previous and allowed to heal, by connecting the proximal and distal ends of the PILOG to the arterial and venous systems of the patient (at different anatomical positions that do not affect or interfere with the repair primary access blood flow) in the manner previously described.

In these and all other scenarios, due to incorporation of most of the length of the PILOG 60 already having occurred, immediate percutaneous needling of the PILOG 60 for dialysis access can occur, thereby removing the need for TUCVC or CVC implantation. After a period of healing of the repaired or revised previously in-use access, the PILOG 60 can be abandoned as use of the primary AVG or primary AVF re-commences. During this period, the PILOG 60 may be placed back into “stand-by mode” after disconnection of the venous and arterial anastomoses or their ligation, by re-inserting a new lumen obturator 3 and securing it in the lumen with a suture or by ligating the ends of the PILOG 60 to entrap the obturator 3 at both ends. This maneuver ensures that embolization or dislodgement of the obturator 3 is prevented and that most of the lumen 1A of the PILOG 60 will remain patent for possible future use.

Alternatively, the use of the PILOG 60 can be continued as it could assume the role of the primary access conduit. In another scenario, and as illustrated in FIG. 6, more than one PILOG 60 could be implanted at the first or at subsequent procedures, each in its own separate and dedicated tissue tract, to afford the patient several ready options for immediately usable dialysis conduits in the event of primary conduit failure, and only requiring relatively simple surgical procedures to enable same-day use of the chosen PILOG 60. As shown in FIG. 6, three PILOGs 60 could be implanted proximal one another, for example. In all conceivable situations, activation of the PILOG 60 as percutaneous access device reduces or removes the requirement for temporary or longer-term dialysis catheter use, and the attendant possible complications associated with the latter.

With all of the above in mind, PILOG implantation may have certain limitations and potential complications worthy of consideration. The PILOG 60 is an implant composed of synthetic materials, and as such is expected to be vulnerable to all of the potential complications usually associated with synthetic implants. Therefore, the PILOG 60 could be extruded or become exposed due to overlying tissue breakdown, or become infected, as a perioperative complication or occurring at a time remote to the implantation procedure, due to hematogenous seeding of bacteria originating from a remote anatomical site, or as a direct consequence of contamination being introduced via the frequent percutaneous needle access events. However, experience with PTFE, Dacron, and implantation-grade medical silicone products has shown a relatively low incidence of the spontaneous development of infections, and even for infection of the blood from other sources to seed such devices to the extent that their explanation becomes necessary. Nevertheless, the incidence is clinically significant but would not be expected to occur at higher rate in the PILOG 60 than in other implants composed of similar materials. After commencement of use as an active dialysis access conduit, the rate of infection, thrombosis, and obstruction of the PILOG 60 due to neo-intimal hyperplasia would be similar to other PTFE conduits currently in established use.

In the case of an infection developing in a primary AVF, or more relevantly, AVG, it often becomes necessary to excise the entire conduit, especially if it is an AVG, to control and adequately treat the infection. Surgical experience has shown however that most often the infection, often suppurative, is confined to the specific conduit tract and surrounding tissues are not directly involved in the suppurative process. Moreover, it is often seen that previously implanted and formerly utilized synthetic grafts, whether present in their entirety or as residual fragments, rarely become foci of infection even when a suppurative infection with or without a blood-culture positive process is occurring in relative proximity to the old graft or its remnants. In this common clinical situation, as would be with an incorporated PILOG 60 with the obturator in situ, no blood is flowing through the prosthesis. As such, it is entirely possible to envisage an infected graft being removed, and after an appropriate period of antibiotic therapy and wound healing, arterial and venous connections being constructed to an incorporated PILOG 60, either in the same limb or in a different portion of the same limb or in another limb entirely with the commencement of percutaneous needling of the PILOG 60 as early as the same day as its activation.

While one or more preferred embodiments are disclosed, many other implementations will occur to one of ordinary skill in the art and are all within the scope of the invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. Other arrangements, methods, modifications, and substitutions by one of ordinary skill in the art are therefore also considered to be within the scope of the present invention, which is not to be limited except by the claims that follow.

While apparatuses and methods are described in terms of “comprising,” “containing,” or “including” various components or steps, the apparatuses and methods can also “consist essentially of” or “consist of” the various components and steps. All numbers and ranges disclosed above may vary by some amount.

Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

What is claimed is:

1. A medical device implantable into a patient, the medical device comprising:
a tube graft defining a lumen; and
an obturator removably seated within the lumen.

2. The medical device of claim 1, further comprising a cord securely coupled to the obturator to form a loop.

3. The medical device of claim 2, wherein the cord is formed from a braided fibrous material.

4. The medical device of claim 3, wherein the fibrous material is nylon.

5. The medical device of claim 1, wherein the lumen is about 6 millimeters to about 8 millimeters in diameter.

6. The medical device of claim 1, wherein the tube graft is formed from a thermoplastic polymer and the obturator is formed from an elastomer.

7. The medical device of claim 6, wherein the thermoplastic polymer is Polytetrafluoroethylene (PTFE) and the elastomer is Silicone.

8. A method of percutaneous needle cannulation hemodialysis, comprising:
implanting the medical device of claim 1 into the patient;
retaining the medical device in situ for a period of time until the medical device is to be used as a dialysis access conduit;
grasping a cord securely coupled to the obturator;
removing the obturator from the tube graft by urging the cord away from the tube graft;
surgically connecting a proximal end of the tube graft to an arterial system of the patient;
surgically connecting a distal end of the tube graft to a venous system of the patient;
percutaneously needling the tube graft to provide dialysis access; and
performing hemodialysis on the patient.

9. The method of claim 8, wherein the medical device is implanted into a dorsal aspect of an arm or forearm of the patient.

10. The method of claim 8, further comprising after performing dialysis:
surgically disconnecting the proximal end and the distal end from the arterial and venous systems of the patient; and
reinserting the obturator into the tube graft.
